(19) United States
(12) Patent Application Publication
(10) Pub. No.: US 2015/0065917 A1
(43) Pub. Date: Mar. 5, 2015

(54) NEEDLE EXTENSION AND RETRACTION MECHANISM FOR A SYRINGE FOR DRAWING BLOOD SAMPLES AND PROVIDED WITH A VACUUM TUBE

(71) Applicant: Daniel Alberto Ierfino, Playa del Carmen Quintana Roo (MX)

(72) Inventor: Daniel Alberto Ierfino, Playa del Carmen Quintana Roo (MX)

(21) Appl. No.: 14/011,379

(22) Filed: Aug. 27, 2013

(51) Int. Cl. A61B 5/15

(52) U.S. Cl. CPC A61B 5/150633
USPC 600/576

(57) ABSTRACT

A needle-extension and retraction device for a syringe for collecting blood samples provided with a vacuum tube includes a needle having a front and rear part. The syringe includes a front portion of a reduced diameter, and a rear portion of a greater diameter, in which the said vacuum tube is housed. The extension and retraction mechanism includes a resilient front member, which further includes a resilient rear member. The resilient front member has a resilient return constant that is slightly less than that of the said rear resilient member. A fastening member having a front end and a rear end is disposed between the resilient front member and the resilient rear member. A sealing member that includes a front face and a rear face, is disposed about the rear resilient return element. The device also includes an actuator means disposed on the rear portion of the syringe.

### FIELD OF THE INVENTION

The present invention relates to an extension and retraction mechanism for a syringe needle for drawing blood samples provided with a vacuum tube.

### BACKGROUND

Currently, various syringe-type devices for drawing blood with a vacuum tube exist, having the needle previously incorporated in the syringe, and having mechanisms for retracting said needle after drawing a blood sample.

Document KR2010020290A, for example, discloses a device for drawing blood with a vacuum system comprising a needle already included in the body of the syringe, connected to a device for its retraction (see arrows) using a spring within the cylindrical body of the syringe. One end of the needle draws the sample and is subsequently retracted during drawing, outside the reach of the operator, while the other end discharges the drawn blood within a vacuum tube.

Document WO00/22979 discloses a device for drawing blood comprising a retractable needle, attached to the collection tube of the syringe, remaining encapsulated after the extraction, and both tube and needle remain unused during said operation. The extraction mechanism is vacuum-operated.

Document WO96/27403 discloses a needle-retraction mechanism in a syringe to prevent accidental contact therewith. The retraction mechanism may be incorporated in a syringe or in a system for collecting blood samples from a patient, and it may be activated manually or by a suction device (plunger) using a trigger or actuator, and includes a spring, which produces the retraction of said mechanism.

Document U.S. Pat. No. 8,109,905 discloses a needle-retraction mechanism in a device for collecting blood samples, in which the needle is previously incorporated, and includes a button that decompresses a spring making it possible to retract the needle into a concealed position.

Document U.S. 2009/0216154 discloses an automatically retractable device for drawing blood samples, wherein the needle is previously incorporated into the core linked to a spring and a blood-collection tube. When taking samples, the said collection tube thrusts the needle forward to pierce a vein and withdraw a sample, and upon completion of the extraction, a hook mechanism provided in the syringe barrel makes it possible to retract the needle outside the reach of the operator, thanks to the action of the return force of the spring.

Document U.S. 2011/0270128 discloses a retractable device for collecting blood samples, wherein the needle is previously incorporated into the syringe, and includes a compressible spring mounted between the needle holder and the front end of the housing, wherein it is contained, and which makes the needle disappear after the collection.

Document U.S. 2011/0166476 discloses a needle assembly that includes a housing with a chamber, a distal end and a proximal end that interact with a container for collecting samples. The assembly comprises a cannula having one end coming into contact with the patient, which the other end does not. The end of the cannula coming into contact with the patient extends at least partially from the distal end of the housing, and the interior of the cannula is in fluid communication with the chamber. The assembly further comprises a needle-retracting mechanism concealing the needle within a frame or shield, whereby at least a part of the chamber is visible to the operator in its retracted position.

Document U.S. Pat. No. 4,892,107 discloses a disposable device for collecting blood samples, comprising a syringe carrying an inner, double-tipped needle, in fluid communication with a vacuum tube, and slidable between two positions, one for extension in order to extract a sample, and the other for retraction inside the sleeve of the syringe.

Document U.S. Pat. No. 7,699,813 discloses a safety mechanism for a needle previously incorporated in a syringe, and sliding within the latter from an initial exposed position to a concealed or protected position, all via a spring mechanism.

Document U.S. Pat. No. 7,811,261 discloses a disposable cover assembly for a syringe needle concealing said needle from view before, during and after the injection procedure. This cover includes a housing and a needle sleeve that retracts and extends the needle by an axial movement within the housing. Document U.S. Pat. No. 8,167,820 B2 discloses a device for obtaining blood samples with a retractable needle, which comprises a tubular adapter with a longitudinally elongated channel. The sleeve of the needle comprises a locking collar moving from a first position to a second position upon complete retraction of the sleeve of the needle. A needle holder is provided in said channel in order to move the needle from a distal orientation, in which the needle is exposed, to a proximal orientation, in which both ends of the needle are concealed within the adapter body. The adapter body comprises two resilient members inside which a connector is provided, on which the ends of both resilient return members are inserted. The needle-retraction mechanism disclosed in this document includes many parts and its assembly is highly complicated. In order to retract the needle, the mechanism uses a trigger operated by a lateral force exerted by the operator.

Document U.S. Pat. No. 5,070,885 discloses a disposable blood-collection device that includes a seal manually movable along the syringe body, without the presence of resilient return means, via a slit or groove cut into the body of the syringe, which includes anchoring areas at its ends. The engaging portion performs locking in different openings that are cut in the front and rear part of the syringe in order to extend and retract the needle, as needed.

Document U.S. Pat. No. 4,900,310 also discloses a blood collection device that includes a double-tipped needle supported by a piston, which is longitudinally slidable by manual operator action, without the presence of resilient return means.

Other documents, e.g., U.S. Pat. No. 7,488,297, U.S. 2009/0,156,963, U.S. 2009/0,259,145, U.S. Pat. No. 7,357,783, AR 056421, JP 2004-113519, JP 10-2010-0020290, U.S. 2012/0,203,138, U.S. 2012/0,238,966 and EP 1,516,585 disclose mechanisms for blood collection with a vacuum tube, with different extraction and/or protection mechanisms, whereby some of them protect the vacuum tubes for blood collection, which comprise, as is shown in the figures, projections at their ends for anchoring the syringes, thus avoiding undesirable displacement thereof.

All the disclosed devices fail to fully protect the operator from accidental puncture by the front and rear ends of a syringe, or from any loss of contaminated fluid through the said syringe. None of them teach or propose the presence of a needle-retraction mechanism with a dual resilient member and a specific return constant in order to protect the operator at the time of retraction. Besides, the mechanism of the invention is sealed, thus preventing contaminated fluids from leaving the syringe and keeping the operator from being contaminated by these fluids, and also, blocks the moving parts so as to increase protection. Using the mechanism of the invention, the operator receives the syringe with the concealed needle and with a single linear movement, the needle is in a position ready for use. Moreover, with this mechanism, unnecessary fatigue of materials is avoided, e.g., of the resilient return means included therein, and it is a substantially more comfortable for the operator, as no cap needs to be unscrewed, and no disposal is needed afterwards.

In this regard, the Applicant has developed a novel needle-retraction mechanism that solves the problems described above.

### SUMMARY

Thus, the object of the present invention is a needle-extension and retraction device for a syringe for collecting blood samples, provided with a vacuum tube and comprises:
a needle with a front and rear part; and
said syringe comprising a front part of a reduced diameter, and a rear part of a greater diameter, in which said vacuum tube is housed;
said extension and retraction mechanism comprising a resilient front member, and further comprising therein:
a resilient rear member, wherein said resilient front member has a resilient return constant that is slightly less than that of said rear resilient member;
a fastening member provided with a front and rear end, disposed between said resilient front member and said resilient rear member;
a sealing member that includes a front face and a rear face, disposed about said rear resilient return element;
and an actuator means disposed on said rear portion of said syringe.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 is a sectional view of a syringe that includes the mechanism of the invention.

FIG. 2 shows the form of the syringe that comprises the mechanism of the invention.

FIG. 3 shows the non-compressed mechanism of the invention.

FIGS. 4A-4D show the process of compressing the mechanism of the invention.

FIGS. 5A to 5D show the forward movement of the sealing member included in the mechanism of the invention.

### DETAILED DESCRIPTION OF THE PREFERRED EMBODIMENTS

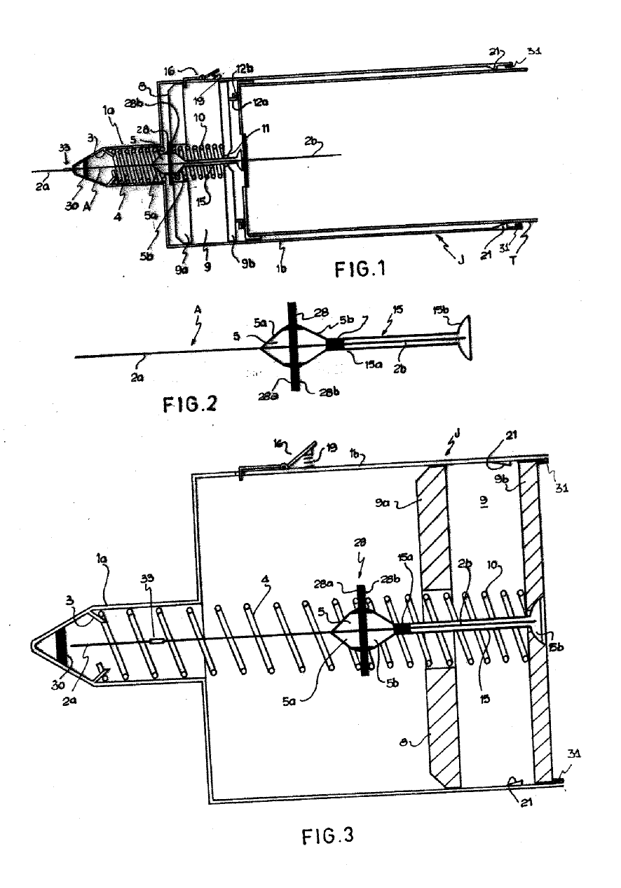

FIG. 1 is a cross section of a syringe J with a vacuum tube T, which includes the mechanism of the invention, and a needle A ready to penetrate a vein and start collecting blood. As is common, said syringe J has a front part 1a of a reduced diameter and a rear part 1b of a greater diameter, in which said vacuum tube T is housed. The mechanism of the invention consists of a pair of resilient members with a specific resilient return constant, a front one labeled 4 and a rear one labeled 10, a fastening member 5 disposed between the two resilient members with a specific resilient return constant, and a sealing member 9 disposed about the rear resilient return member 10, whereby said sealing member 9 is retractable with an actuating means 16. Said sealing member 9 is formed by a front face 9a and a rear face 9b spaced apart from one another. Optionally, and from the outer surface of said rear face 9b, first elements of catch 12a of the half revolution type interacting with second catch members 12b of the half revolution type on the bottom face of said vacuum tube T may extend. These catch members 12a, 12b of the half revolution type allow said vacuum tube T to remain threaded into said syringe (J). FIG. 2 is a detailed depiction of the form of syringe A. Between its front 2a and rear part 2b, a fastening member 5 traversed by said needle A is included. The front end 5a and rear end 5b of said fastening member 5 assume a slightly conical shape in order to secure said pair of resilient return members 4, 10 with different resilient return constants, which members henceforth will be referred to as front spring 4 and rear spring 10. Between said front 5a and rear 5b ends of said fastening member 5, a stop member 28 with a front face 28a and a rear face 28b is provided. The rear part 2b of said needle A is covered by a rubber valve 15. Said rubber valve 15 has a front end 15a and a rear end 15b, the latter with a form resembling a cup or a golf tee, the purpose of which will be explained in the following text. Said front end 15a of said rubber valve 15 engages under pressure a protrusion 7 projecting from the rear end 5b of said fastening member 5, thus preventing its movement. The purpose of said rubber valve 15 is to prevent blood from escaping when the front part 2a of the needle A penetrates a patient's vein, and seal with its cup-shaped rear end 15b a concave recess 11 situated in the rear face 9b of said sealing member 9, whereby said concave recess 11 matches said cup shape of said rear end 15b of said rubber valve 15 and is of greater height than the latter, whereby said rear end 15b is housed slightly below the level of the rear face 9b of said sealing member 9, upon retraction of needle A.

By housing the rear end 15b slightly below the level of the rear face 9b, greater protection is afforded, since upon retraction of needle A, rubber valve 15 cannot be removed by anyone with their fingers. Furthermore, and for better operator protection, said rubber valve 15 remains secured to the rear part 2b of needle A, and the latter is disposed, in turn, within said front 4 and rear 10 springs, making its removal even more difficult. Said rear end 15b of said rubber valve 15 rests on the front face of said vacuum tube T, the latter being traversed by the rear part 2b of said needle A in order to collect blood, as shown in FIG. 1. During extraction, said vacuum tube T may possibly slide backward due to the thrust of rubber valve 15, hence it may be threaded into the syringe J using the above-mentioned first and second catch means 12a, 12b of the half-revolution type, as an operator option. Likewise, as shown also in FIG. 1, said front spring 4 is secured at one end to the front portion of syringe J by projections extending at an angle inside said front spring 4, and at the other end, to the tapered front end 5a of said fastening member 5. Said rear spring 10 is secured at one end to the tapered rear end 5b of fastening member 5, while the other end is supported directly on the inside of the rear face 9b of said sealing member 9.

As seen in FIGS. 1 and 3, the front face 9a of sealing element 9 takes the form of a trapezoid, wherein the lower base 8 rests on said rear face 28b of said stop member 28. Syringe J is supplied to the operator with needle A concealed and the mechanism of the invention decompressed, as shown in FIG. 3. Therefore, at the time of use, sealing member 9 should be moved forward thereby compressing both front spring 4 and rear spring 10. During the compression and decompression process, said actuating means 16 performs an essential role. Said actuating means 16 takes the form of a lever pivoting about its midpoint. This design causes said actuator means 16 to be more comfortable, immediate and smooth to operate compared with prior-art actuator means for syringes, as it prevents unwanted movement of syringe J, once needle A is inside the vein. Specifically, front end 16a of said actuating means 16 penetrates the body of syringe J, preferably perpendicularly thereto.

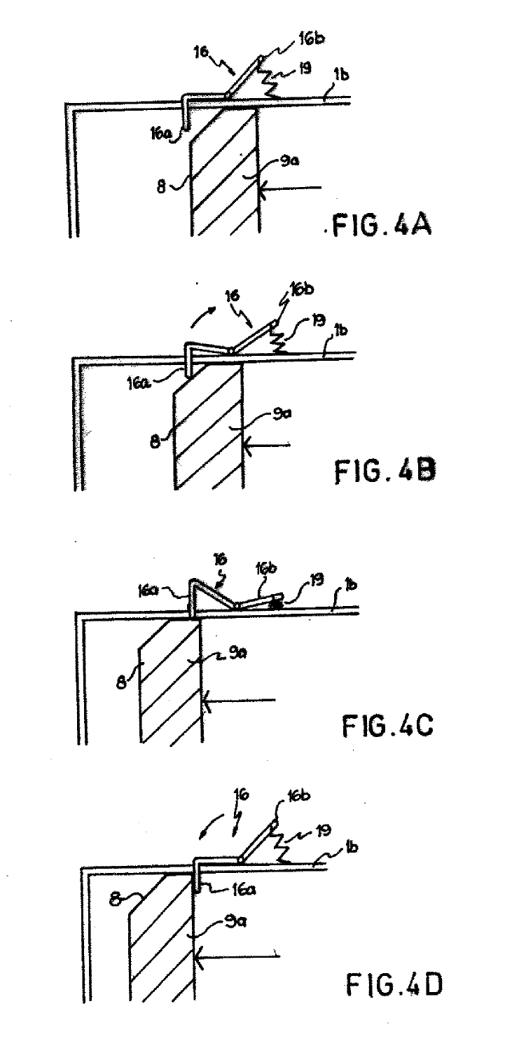

Rear end 16b of said actuation means protrudes from said body of said syringe J, linking to it via a resilient member of actuator 19, which may be a spring. When moving sealing member 9 forward (the compression process), front 4 and rear 10 springs are compressed and front face 9a of said sealing member 9 approaches front end 16a of said actuator means 16, as shown in FIG. 4A. As shown in FIG. 4B, the smaller base 8 of said front face 9a of said sealing member 9 reaches a point where, due to its trapezoidal configuration, it begins to advance the front end 16a of said actuating means 16, thereby compressing spring 19, see FIG. 4B, until leaving the inside the body of syringe J, see FIG. 4C. When the front face 9a of said sealing member 9 passes fully below the front end 16a of said actuating means 16, said front end 16a, due to the return action of compressed spring 19 now being released, returns to penetrate into the body of the syringe J, automatically locking said sealing member 9 behind the front face 9a, thereby preventing said sealing means 9 from receding, due to the action of said front 4 and rear 10 springs, see FIG. 4D. Said actuating means 16 should be situated somewhere in the rear portion 1b of said syringe J, such that the catch of said sealing member 9 causes the front face 9a to be in the proximity of the front portion of said syringe J and keeps said needle A exposed.

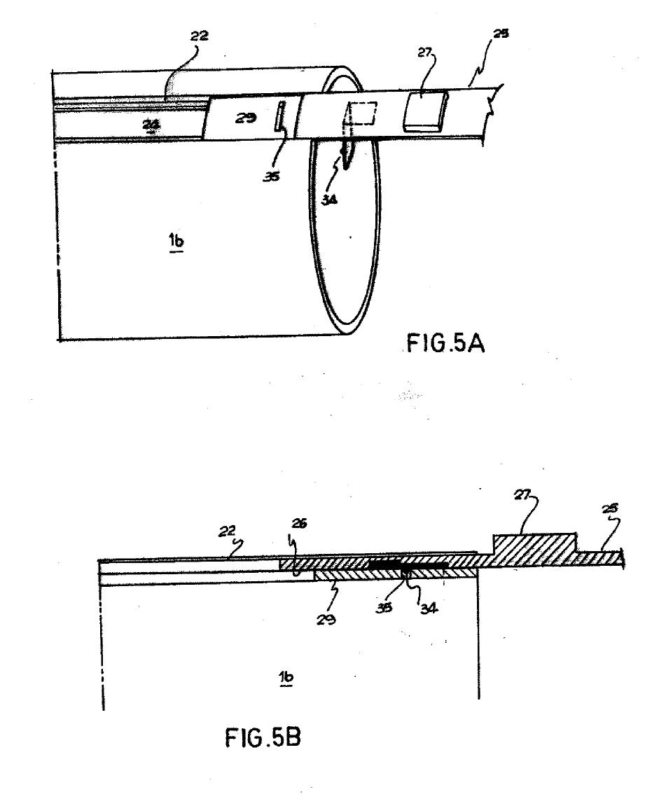

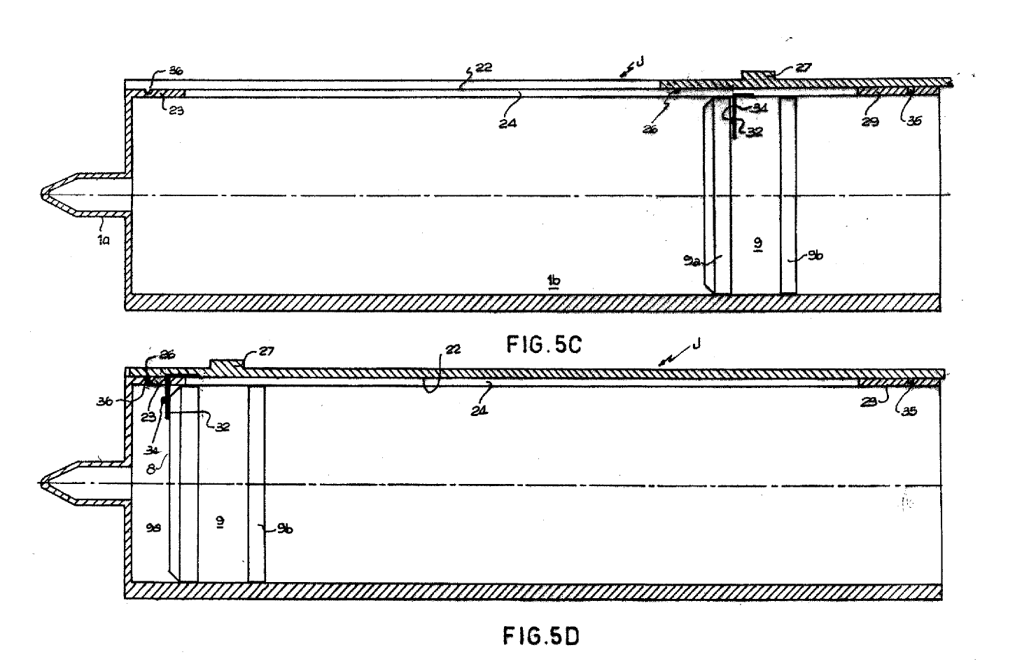

The exposed portion of said needle A may include a small portion 33, just a few millimeters long (see FIG. 1), of transparent material, allowing the operator to ascertain that needle A is in a vein, as the said small portion 33 will turn red, when this occurs. The forward movement of said sealing means 9 in order that said needle A remain exposed and the front face 9a of said sealing member 9 remain situated in the proximity of the front portion of said syringe J is described in the following. FIG. 5A shows that the rear portion 1b of said syringe J includes a longitudinal depression 29 which includes a first transverse groove 35. Moreover, and as shown in FIGS. 5C, the front of the rear portion 1b of said syringe J includes a rectangular space 23 with a second transverse groove 36. Between said rectangular space 23 and said longitudinal depression 29, a very narrow longitudinal cutout 24 is made, with guides 22 on either side thereof, and in which, a bar 25 moves, which is preferably made of plastic material. Returning to FIG. 5A, said bar 25 comprises a protrusion 27 on top in order to facilitate its movement by the operator's fingers.

Furthermore, said bar 25 comprises on its bottom a small flexible portion 32, made preferably of a plastic material, which adopts a position perpendicular to said bar 25 when not flexed. Said flexible part 32, in turn, includes somewhere on its surface a horizontal safety catch 34 interacting with said first transverse groove 35. As shown in FIG. 5B, syringe J is originally supplied with said flexible portion 32 bent over said bar 25, with said horizontal safety catch 34 engaged in said first transverse groove 35 to prevent any accidental puncture when transported or handled. The operator may start moving the bar 25 by means of protrusion 27, or simply by pushing from the start of bar 25. To generate such a movement, the operator must exert a certain force releasing said horizontal safety catch 34 of said first transverse groove 35, re-attaining the perpendicular position against said bar 25.

Subsequently, said flexible portion 32 rests on the rear surface of front face 9a of sealing element 9, as shown in FIG. 5C. After which, said bar 25 pulls said sealing member 9 forward, compressing the front 4 and rear 10 springs. As the bar 25 is moved forward by the operator, so is sealing member 9. The operator may continue to push bar 25, including when sealing member 9 has reached the end of its travel, i.e. when said front 4 and rear 10 springs are fully compressed, as shown in FIG. 5D. This additional pressure will make the flexible portion 32 disengage from said sealing member 9, again flexing over said bar 25, causing said bar 25 to continue moving forward.

Finally, the front end of said bar 25 is inserted into said second inner groove 36 of said rectangular space 23 by means of a tip 26, such that said bar 25, at the end of its travel, does not protrude in front of or in the back of said rear portion lb of said syringe J, thus remaining immobile and preventing this part from opening. Said bar 25 has the same curvature as the body of the syringe J affording it more firmness, so that it does not easily bend. Retraction of sealing element 9 (the decompression process) occurs, when the operator presses actuating means 16, thereby releasing the compression of the front 4 and rear springs. In this way, said front spring 4 has a return constant (called k) that is slightly less than that of the rear spring 10, such that upon retraction, sealing member 9 exceeds the rear part 2b of needle A, while the rear end 15b of said rubber valve 15 attaches in said concave recess 11 of said rear face 9b of said sealing member 9, thereby protecting the operator from an accidental puncture during retraction.

The combined thrust of springs 4 and 10 upon sealing element 9 causes the latter to momentarily push and compress slightly the tracks of safety catches 21 (see FIGS. 1 and 3) situated inside the top end of the rear portion 1b of said syringe J, in order to move beyond them and subsequently become embedded in the part situated between the front 9a and rear 9b face of said sealing member 9, thereby preventing it from being moved forward again. Stop and sealing members 31 disposed in continuation of said safety catches 21 (see FIGS. 1 and 3), including inside said rear portion 1b of said syringe J, and at a distance of said safety catches 21 that matches the thickness of the rear face 9b of said sealing member 9, eventually fix said sealing member 9 within the body of the syringe J, preventing the body from being moved, e.g., upward. Thus, the sealing is absolute and besides, the mechanism of the invention remains completely immobile, thereby preventing any kind of accident due to puncture or contamination of the fluids. In addition, the tip of the front portion of the syringe J includes a membrane 30 in order to prevent any fluid from escaping.

It is understood, therefore, that this invention is not limited to the particular embodiments disclosed, but is intended to cover all modifications which are within the spirit and scope of the invention as defined by the appended claims; the above description; and/or shown in the attached drawings.

1. A needle-extension and retraction device for a syringe (J) for collecting blood samples provided with a vacuum tube (T), comprising:
a needle (A) having a front (2a) and rear part (2a); and
said syringe (J) comprising a front portion (1a) of a reduced diameter and a rear portion (1b) of a greater diameter, in which said vacuum tube (T) is housed;
said extension and retraction mechanism comprising a resilient front member (4) and a resilient rear member (10), wherein said resilient front element (4) has a resilient return constant that is slightly less than that of said rear resilient member (10);
a fastening member (5) provided with a front end (5a) and a rear end (5b), disposed between said resilient front member (4) and said resilient rear member (10);
a sealing member (9) that includes a front face and a rear face, disposed about said rear resilient return element (10); and
an actuator means (16) disposed on said rear portion (1b) of said syringe (J).

2. A mechanism according to claim 1, wherein said fastening member (5) is traversed by said needle (A).

3. A mechanism according to claim 1, wherein said front (5a) and rear (5b) ends of said fastening member (5) adopt a slightly conical shape in order to secure said front and rear resilient members (4, 10).

4. A mechanism according to claim 3, wherein said resilient front (4) and rear (10) members are springs.

5. A mechanism according to claim 1, wherein between said front (5a) and rear (5b) ends of said fastening member (5), a stop element (28) having a front face (28a) and a rear face (28b) is provided.

6. A mechanism according to claim 5, wherein said rear face (28a) rests on said front face (9a) of said sealing member (9).

7. A mechanism according to claim 1, wherein the rear part (2b) of said needle (A) is covered by a rubber valve (15).

8. A mechanism according to claim 7, wherein said rubber valve (15) has a front end (15a) and a rear end (15b), whereby said front end (15a) engages under pressure a protrusion (7) extending from the rear end (5b) of said fastening member (5), and said rear end (15b) adopts a shape resembling a cup or a golf tee.

9. A mechanism according to claim 1, wherein said rear face (9b) of said sealing member (9) includes a concave recess (11) matching the shape of said cup-shaped rear end (15b) of said rubber valve (15) and of greater height than said rear end (15b) of said rubber valve (15).

10. A mechanism according to claim 7, wherein said rear end (15b) of said rubber valve (15) rests on the front face of said vacuum tube (T), the latter being traversed by said rear part (2b) of said needle (A).

11. A mechanism according to claim 1, wherein said front spring (4) is secured at one end to said front portion of said syringe (J) by projections (3) that extend at an angle into said front spring (4) and at the other end to the tapered front end (5a) of said fastening member (5).

12. A mechanism according to claim 1, wherein said rear spring (10) is secured at one end to the tapered rear end (5b) of said fastening member (5), and the other end rests directly on the inner part of the rear face (9b) of said sealing member (9).

13. A mechanism according to claim 1, wherein said front face (9a) of said sealing member (9) takes the form of a trapezoid, in which the lower base (8) rests on said rear face (28b) of said stop element (28).

14. A mechanism according to claim 1, wherein said actuator means (16) takes the form of a lever that pivots about its midpoint, whereby the front end (16a) of said actuator means (16) penetrates in a perpendicular fashion the said syringe body (J) in order to lock said shutter member (9), and the rear end (16b) of said actuator means (16) protrudes from said body of said syringe (J), linking thereto via a resilient member of the actuator (19), said actuator means (16) being disposed somewhere on the rear portion (1b) of said syringe (J), such that by locking said sealing member (9), said face (9a) said sealing member (9) will become disposed in the proximity of the front portion (1a) of said syringe (J), and such that said needle (A) is exposed.

15. A mechanism according to claim 14, wherein said resilient member of actuator (19) is a spring.

16. A mechanism according to claim 14, wherein the exposed portion of said needle (A) includes a small transparent portion (33), just a few millimeters long, allowing the operator to ascertain that said needle (A) is in a vein.

17. A mechanism according to claim 1, wherein the rear part of said rear portion (1b) of said syringe (J) includes a longitudinal depression (29) that includes a first transverse groove (35), and the front portion posterior (1b) of the syringe (J) includes a rectangular space (23) with a second transverse groove (36), whereby between said rectangular space (23) and said longitudinal depression (29), a very narrow longitudinal cutout (24) with guides (22) on each side thereof is made, and upon which a bar (25) moves, preferably made of a plastic material, whose front end includes a tip (26) interacting with said second transverse groove (36).

18. A mechanism according to claim 17, wherein said bar (25) includes a protrusion (27) on top in order to facilitate movement thereof by the operator's fingers, and a small flexible part (32) in its bottom, which adopts a position perpendicular to said bar (25), when not flexed.

19. A mechanism according to claim 18, wherein said flexible portion (32), in turn, includes, somewhere on its surface, a horizontal safety catch (34) interacting with said first transverse slot (35).

20. A mechanism according to claim 17, wherein said syringe (J) is originally supplied with said flexible portion (32) flexed over said bar (25), with said horizontal safety catch (34) engaged in said first transverse slot (35) to prevent any accidental puncture, when transported or handled.

21. A mechanism according to claim 17, wherein at the end of the travel of said bar (25), said tip (26) engages in said second inner groove (36), such that said bar (25) does not protrude in front of or in the back of said rear portion (1b) of said syringe (J).

22. A mechanism according to claim 17, wherein said bar (25) has a same curvature as said syringe body (J).

23. A mechanism according to claim 1, wherein the inside of the top end of the rear portion (1b) of said syringe (J) includes safety catches (21) and stop and sealing members (31) disposed in continuation of said safety catches (21), at a distance of these safety catches (21) matching the thickness of said rear face (9b) of said sealing member (9).

24. A mechanism according to claim 1, wherein the tip of said front portion (1a) of said syringe (J) includes a membrane (30) in order to prevent any fluid from escaping.

25. A mechanism according to claim 1, wherein from the outer surface of said rear face (9b), first catching members (12a) of the half-revolution type interacting with second catching elements (12b) of the half-revolution type situated in the lower face of said vacuum tube (T) extend, such that said vacuum tube (T) is threaded into said syringe (J).
